(19) United States
(12) Patent Application Publication
(10) Pub. No.: US 2023/0015005 A1
(43) Pub. Date: Jan. 19, 2023

(54) METHOD, APPARATUS AND COMPUTER-READABLE MEDIUM OF SENIOR CLOUD SERVICE FOR HEALTH CARE

(71) Applicant: HUMAN IT SOLUTION CO., LTD., Mokpo-si (KR)

(72) Inventors: Bongdu CHOI, Mokpo-si (KR); Hosung PARK, Changwon-si (KR); Green BANG, Gimpo-si (KR)

(21) Appl. No.: 17/844,188

(22) Filed: Jun. 20, 2022

(30) Foreign Application Priority Data
Jul. 16, 2021 (KR) 10-2021-0093336

(51) Int. Cl. G09B 19/00; G16H 40/20; G06Q 50/22

(52) U.S. Cl. CPC G09B 19/00; G16H 40/20; G06Q 50/22

(57) ABSTRACT

Disclosed is a method of a senior cloud service for health care, which including: a training center search step of searching for a training center for performing a training scheduled in a training curriculum set in a trainee account based on location information of a trainee terminal, when a health care service request is received from the trainee account; an evaluation result collection step of providing a search result of the training center to the trainer account, and collecting an evaluation result for training details performed by a trainee from a terminal of the training center determined as generating record of a visit record of the trainee account; and an update step of updating a degree of achievement of training curriculum set in the trainee account and a degree of training performance previously stored in the trainee account by using the collected evaluation result.

### BACKGROUND OF THE INVENTION

### 1. Field of the Invention

The present invention relates to a method of a senior cloud service for health care, and more particularly, to a technology for providing a health care service using on-site infrastructure through an application installed on a terminal of a trainee, and allowing a cloud server to integrally manage details of the health care service used by the trainee.

### 2. Description of the Related Art

Dementia refers to a complex symptom in which the normally mature brain is damaged or destroyed by external factors such as acquired trauma or disease, and deteriorated in overall cognitive functions, such as intelligence, learning and language, and higher mental functions.

Unfortunately, there is currently no definitive treatment for dementia, and it is expected that social and economic burdens caused by dementia will increase. Accordingly, the needs have been increased day by day to prevent dementia or implement care environments for safely training dementia patients while reducing mental and physical burdens of supporters of the dementia patients.

As a related art, Korean Patent Registration No. 10-2266310 (METHOD, APPARATUS AND COMPUTER-READABLE MEDIUM OF ARTIFICIAL INTELLIGENCE BASED INSTRUCTIONAL CONTENT RECOMMENDATION FOR DEMENTIA CARE ENHANCING COGNITIVE ABILITY) discloses the technology in which an cognitive ability evaluation is performed for a large number of trainees unfamiliar with dementia care services, and areas required for reinforcing or improving the cognitive ability of the trainee are identified from the previous cognitive ability evaluation results of the trainee, so that customized educational content for improving the cognitive ability is planned and provided as a single curriculum.

Meanwhile, according to the above-described related art, there is a positive function of implementing a more systematic training service by composing a training curriculum and a training content set with educational contents for improving the cognitive ability of a trainee, but there is a significant limitation in time and place since the trainee is required to visit a designated training center to conduct a training according to the training curriculum.

Accordingly, in order to increase the positive function of the above-described related art, there are needs to develop technology for maximizing the nearby on-site infrastructure based on the location of the trainee so as to assist the trainee in conducting a training.

### SUMMARY OF THE INVENTION

Accordingly, the present invention has a first object for providing a health care service using on-site infrastructure through an application installed on a terminal of a trainee, and allowing a cloud server to integrally manage details of the health care service used by the trainee.

In addition, the present invention has a second object for guiding places for using health care services while considering a trainee's preset training curriculum so as to increase access to health care services.

In order to achieve the above objects, a method of a senior cloud service for health care according to one embodiment of the present invention according to one embodiment of the present invention implemented by a computing device including at least one processor and at least one memory for storing instructions executable in the processor includes: a training center search step of, when a health care service request is received from a trainee account through a health care application installed on a trainee terminal, searching for at least one training center, which performs a training scheduled in a training curriculum set in the trainee account, based on location information of the trainee terminal; an evaluation result collection step of providing a search result of the training center to the trainer account, and collecting an evaluation result for training details performed by a trainee from a terminal of the training center determined as generating record of a visit record of the trainee account; and an update step of updating a degree of achievement of the training curriculum preset in the trainee account and a degree of training performance previously stored in the trainee account, by using the collected evaluation result, wherein at least one data processed in the training center search step, the evaluation result collection step, and the update step is managed through a cloud server linked to the health care application.

In the training center search step, location information including at least one of a current location of a terminal accessing the trainee account and a location selected in the trainee account, and training performance information required to be obtained from the trainee account may be weighted to provide a matching training center as a first search result, in which the training performance information may be preferably an input value for a training set of a unit training session scheduled in the training curriculum.

In addition, in the evaluation result collection step, the collection of evaluation results may be pended when the trainee is determined as having visited the training center included in the first search result and performed a training but a training set of the unit training session scheduled in the training curriculum at the training center is determined as having been finished in an incomplete state, and it may be preferable, upon next training center search step, to provide a training center as a second search result that matches location information, training performance information and the training set of the unit training session finished in the incomplete state.

In addition, when it is determined that the above-described trainee has visited a training center included in the second search result, it is preferable that the computing device may transmit an instruction of performing in advance the training set of the unit training session finished in the incomplete state in the trainer account to a terminal of the training center, so as to perform collection of the pending evaluation results and then allow training corresponding to a next training session in the training curriculum set in the trainee account to proceed.

In addition, in the training center search step, when a plurality of trainings are scheduled in the training curriculum set in the trainee account, and a search result for a single training center capable of performing the trainings is not found, it may be preferable to provide search results grouped by two or more training centers by searching for two or more training centers capable of performing an individual training included in the trainings.

In addition, it may be preferable that an accessibility index from the trainer terminal may be indicated in the search results grouped into the above-described two or more training centers, in which the accessibility index may be indicated preferentially in the trainer terminal after a higher weight may be applied to a smaller number of training centers that the trainee is required to use and a shorter moving distance between the training centers.

Meanwhile, an apparatus of a senior cloud service for health care, which implemented by a computing device including at least one processor and at least one memory for storing instructions executable in the processor includes: a training center search unit for searching for at least one training center, which may perform a training scheduled in a training curriculum set in the trainee account, based on location information of the trainee terminal when a health care service request is received from a trainee account through a health care application installed on a trainee terminal; an evaluation result collection unit for providing a search result of the training center to the trainer account, and collecting an evaluation result for training details performed by a trainee from a terminal of the training center determined as generating record of a visit record of the trainee account; and an update unit for updating a degree of achievement of the training curriculum preset in the trainee account and a degree of training performance previously stored in the trainee account, by using the collected evaluation result, wherein at least one data processed in the training center search unit, the evaluation result collection unit, and the update unit is managed through a cloud server linked to the health care application.

On the other hand, the present invention provides a computer-readable recording medium that stores instructions for allowing a computing device to perform the following steps including: a training center search step of searching for at least one training center, which performs a training scheduled in a training curriculum set in a trainee account, based on location information of a trainee terminal, when a health care service request is received from the trainee account through a health care application installed on the trainee terminal; an evaluation result collection step of providing a search result of the training center to the trainer account, and collecting an evaluation result for training details performed by a trainee from a terminal of the training center determined as generating record of a visit record of the trainee account; and by using the collected evaluation result an update step of updating a degree of achievement of the training curriculum preset in the trainee account and a degree of training performance previously stored in the trainee account, wherein at least one data processed in the training center search step, the evaluation result collection step, and the update step is managed through a cloud server linked to the health care application.

According to one embodiment of the present invention, the health care application installed on the trainee terminal is used, so that the health care services utilizing on-site infrastructure near the trainee terminal can be provided.

In addition, according to the present invention, the details and evaluation results of the trainee are collected from the terminals of the training center and synchronized with the cloud server in real time, so that the cloud-based health care services, such as unifying and providing multiple databases at a quick response speed, can be provided.

In addition, according to one embodiment of the present invention, places for using health care services are guided while considering a trainee's preset training curriculum to increase accessibility to the health care services, so that the efficiency of improving the cognitive ability of the trainee can be increased, and the convenience for the trainee using the training center can be remarkably increased.

### BRIEF DESCRIPTION OF THE DRAWINGS

FIG. 1 is a flow chart of a method of a senior cloud service for health care according to one embodiment of the present invention.

FIGS. 2 to 4 are examples in which search results of training centers are provided according to one embodiment of the present invention.

FIG. 5 is a conceptual diagram showing that evaluation results of a trainee are collected at each training center terminal to update the training curriculum achievement and the training performance of the trainee according to one embodiment of the present invention.

FIG. 6 is an example in which the training curriculum achievement and the training performance of the trainee are provided according to one embodiment of the present invention.

FIG. 7 a configuration diagram of an apparatus of a senior cloud service for health care according to one embodiment of the present invention.

FIG. 8 shows one example of an internal configuration of a computing device according to one embodiment of the present invention.

### DETAILED DESCRIPTION OF THE INVENTION

Hereinafter, various embodiments and/or aspects will be described with reference to the drawings. In the following description, numerous specific details are set forth in order to provide a thorough understanding of one or more aspects for the purpose of explanation. However, it will also be appreciated by a person having ordinary skill in the art that such aspect(s) may be carried out without the specific details. The following description and accompanying drawings will be set forth in detail for specific illustrative aspects among one or more aspects. However, the aspects are merely illustrative, some of various ways among principles of the various aspects may be employed, and the descriptions set forth herein are intended to include all the various aspects and equivalents thereof.

The term “embodiment”, “example”, “aspect”, “exemplification”, or the like as used herein may not be construed in that an aspect or design set forth herein is preferable or advantageous than other aspects or designs.

In addition, it will be understood that the terms “include” and/or “comprise” specify the presence of the corresponding feature and/or element, but do not preclude the possibility of the presence or addition of one or more other features, elements or combinations thereof.

In addition, the terms including an ordinal number such as first and second may be used to describe various elements, however, the elements are not limited by the terms. The terms are used only for the purpose of distinguishing one element from another element. For example, the first element may be referred to as the second element without departing from the scope of the present invention, and similarly, the second element may also be referred to as the first element. The term “and/or” includes any one of a plurality of related listed items or a combination thereof.

In addition, unless defined otherwise in the embodiments of the present invention, all terms used herein including technical or scientific terms have the same meaning as commonly understood by those having ordinary skill in the art. Terms such as those defined in generally used dictionaries will be interpreted to have the meaning consistent with the meaning in the context of the related art, and will not be interpreted as an ideal or excessively formal meaning unless expressly defined in an embodiment of the present invention.

The present invention relates to a method of a senior cloud service for health care. The present invention has a first object for providing a health care service using on-site infrastructure through an application installed on a terminal of a trainee, and allowing a cloud server to integrally manage details of the health care service used by the trainee, and has a second object for guiding places for using health care services while considering a trainee's preset training curriculum so as to increase access to health care services.

Hereinafter, the present invention will be described with reference to the accompanying drawings as a description of the present invention for achieving the above objects, and a plurality of drawings may be simultaneously referenced in order to describe one or more technical features or components constituting the present invention.

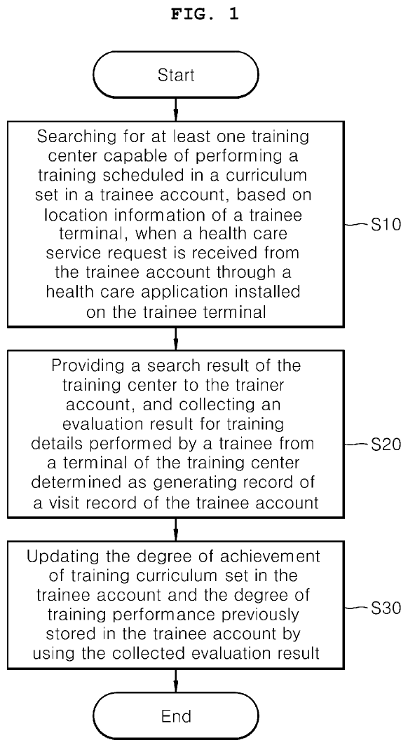

First, referring to FIG. 1, FIG. 1 shows a flow chart of a method of a senior cloud service for health care according to one embodiment of the present invention.

As shown in FIG. 1, the present invention includes performing a training center search step S10 of searching for at least one training center capable of performing a training scheduled in a curriculum set in a trainee account, based on location information of a trainee terminal, when a health care service request is received from the trainee account through a health care application (APP) installed on the trainee terminal.

The health care application may be understood as a concept of application software that collectively manages the trainee's training plan, training status, and training results, and preferably, may be understood that even biological change values during training the trainee is recorded and managed in conjunction with training tools of the trainee.

Specifically, in the above-described step S10, at least one training center for performing a training scheduled in a training curriculum set in the trainee account is searched based on location information of the trainee terminal. The training center may be understood as a concept of a place including at least one of a rehabilitation medical department, a welfare center, and a rehabilitation training center, and the health care service provided by the training center may be preferably understood as a concept of training for cognitive enhancement for preventing dementia.

As one example, in the above-described step S10, the above-described training center may be searched based on location information including at least one of a current location of a terminal accessing the trainee account and a location selected in the trainee account.

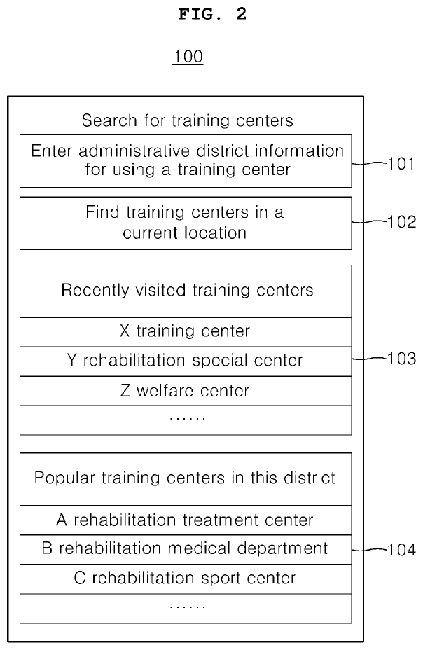

Referring to FIG. 2 simultaneously as a more specific example, it can be seen that 100 of FIG. 2 shows an example of an interface for guiding the search for training centers to the trainee account accessing the health care application in order to receive the search results on training centers.

As indicated in 101 of FIG. 2, according to the present invention, information on an administrative district to be used for a training center is allowed to be inputted to the trainee account through an input mechanism of the trainee terminal, so that search results on training centers corresponding to the input value may be provided.

In addition, as indicated 102 in FIG. 2 according to the present invention, location information where the trainee terminal is currently located is received from a GPS module provided in the trainee terminal, so that the case where the trainee has no address information on the administrative district may be responded.

In addition, as indicated 103 and 104 in FIG. 2 according to the present invention, when a record of using the training center is present in the trainer account, information on recently visited training centers may be displayed, or a state of using training centers in a unit district where the trainee terminal is currently located may be analyzed in the cloud server, so as to mark and guide popular training centers in the district.

In other words, according to the above embodiments in the present invention, the convenience of the trainee can be remarkably increased in searching for training centers.

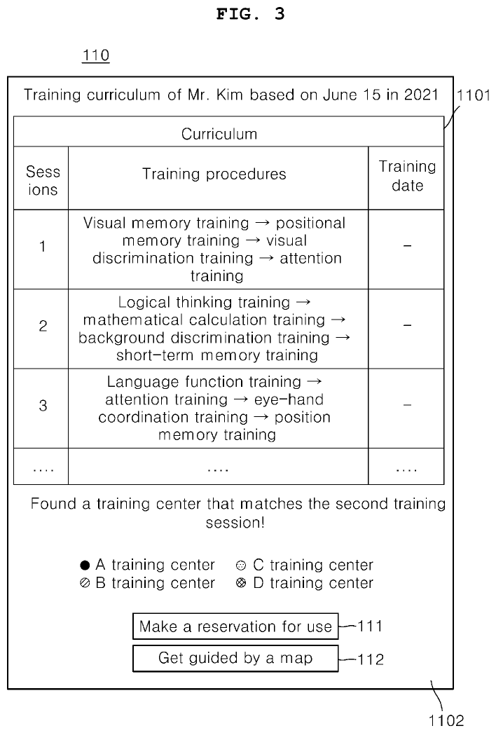

Referring to FIG. 3 as an example, 110 of FIG. 3 shows an example that provides a search result 1102 on a training center matching and corresponding to a training curriculum 1101 set in the trainee account.

Meanwhile, according to the present invention, an option (111) of reservation for use and an option (112) of guide by map may be implemented in an interface displayed in the trainer account in addition to simply providing the search result 1102 on the training centers, so as to perform a reservation for use taking into account the visiting date and time to the found training center in advance, or perform a function to receive a location guidance on a map in detail. However, the present invention is not limited thereto.

The above-mentioned reservation may also be understood that a proxy reservation is allowed in a guardian terminal registered in the trainee account, in view of the difficulty in directly making a reservation by an older trainee. However, the present invention is not limited thereto.

Returning back to FIG. 1, another embodiment of the step S10 will be described. According to the present invention, in addition to simply providing the search results on the training centers based on the location of the trainee terminal upon searching for training centers, training performance information to be obtained from the trainee account may be given as a weight to provide a matching training center as a first search result.

The training performance information may be an input value for a training set of a unit training session scheduled in the training curriculum.

More specifically according to the present invention, each training curriculum may be planned in the trainee account based on evaluation results on trainee cognitive ability, the training curriculum may be composed of individual unit training sessions, and at least one training content for each event for reinforcing the trainee cognitive ability may be set in the unit training session.

Meanwhile, according to the present invention, the training content for each event included in one unit session may be defined as one training set, and as a result, the above-described training performance information may be understood as selection input information for a training set (training content for each event included in the curriculum) selected in the trainee account.

In other words, upon providing the search results on training centers according to the present invention, the exposure priority of the search results may be determined by giving a weight to the location of the trainee and the training performance information that the trainee wants to obtain, that is, by giving a weight to a training center close to the trainee and capable of performing a training for the performance that the trainee wants to achieve.

According to the present invention, when the training center capable of perform the training for the performance that the trainee wants to achieve is not found, it may be desirable to provide search results in which a search range for the training centers is gradually expanded to a preset critical distance radius.

Meanwhile, as another embodiment of the step S10 according to the present invention, when a plurality of training sets are scheduled in the training curriculum set in the trainee account, and it is desirable that a moving route of the trainee may be reduced by searching for a single training center capable of performing the training sets so as to increase the convenience of the trainee.

However, upon performing step S10, and when a search result for a single training center capable of performing the training set is not found, search results grouped by two or more training centers may be provided by searching for two or more training centers capable of performing an individual training included in the training sets, thereby remarkably utilizing the on-site infrastructure of the unit district, so that the accessibility of the trainee to the training center can be increased.

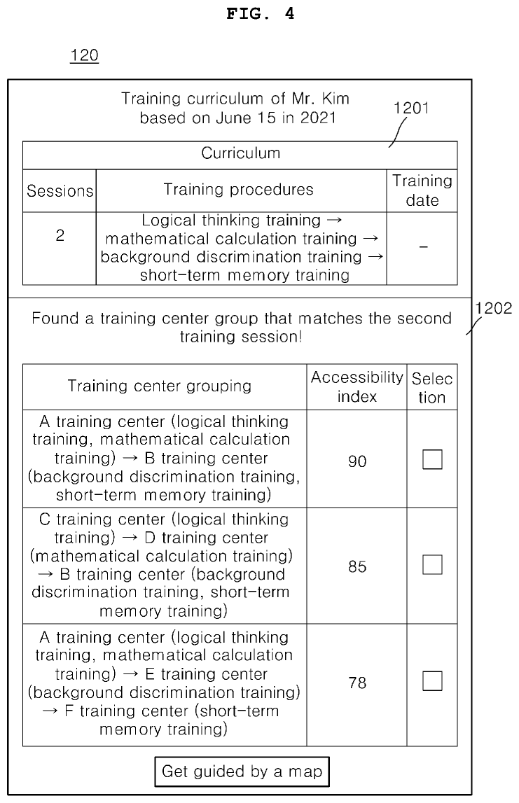

As one embodiment, 120 of FIG. 4 shows an example that indicates information on the training scheduled in the training curriculum 1201 of the trainee, and provides search results grouped into two or more training centers as search results 1202.

In other words, it may be understood that the grouped search result 1202 according to the present invention may be provided with a list in the trainee account while connecting two or more training centers capable of providing health care services for trainees' training contents.

As shown in FIG. 4 according to the present invention, it is preferable to group the training centers corresponding to a training sequence while taking into consideration the training sequence in the training curriculum.

In other words, this is a search process of training centers while considering that the training sequence of the training curriculum may have a significant effect on the improvement of the trainee cognitive ability. Accordingly, the health care services thoroughly subject to the training curriculum can be provided.

Further, in a more preferred embodiment of the present invention, as shown in 1202 of FIG. 4, the convenience of selecting a training center group may be provided to the trainee by indicating the accessibility index from the trainee terminal in the search results grouped into two or more training centers.

The accessibility index may be understood that the convenience of the trainee using the training center is expressed as a quantitative numerical value, which is good or bad. In the grouped search results, a higher weight may be given when the number of training centers used by the trainee is smaller (that is, when the number of routed training centers is smaller) and a moving distance between the training centers is shorter.

As more specific example, the accessibility index may be expressed numerically from 0 to 100. In the case of setting the default accessibility index to 50, +50 is given when the number of training centers is 2 or less, +35 is given when more than 2 and less than or equal to 4, and +20 is given when more than 4. In addition, a score may be given, for example, +50 is applied when the distance between training centers is less than 200 m, +35 is applied when more than 200 m and less than or equal to 500 m, and +20 is applied when more than 500 m and less than or equal to 800 m. An average between the score calculated by the number of training centers and the score calculated by the distance of training centers is applied to the accessibility index, so that the accessibility index provided to the user account may be calculated.

In other words, the accessibility index may be calculated as the following formula: Accessibility index=Basic accessibility index+((calculated score according to the number of training centers)+(calculated score according to the distance of training centers)/2)

Meanwhile, regarding the accessibility index calculated in the above manner, the higher accessibility index may be indicated with priority as shown in 1202 of FIG. 4, thereby functioning to provide the trainee with an optimal access route to the training centers, so that the satisfaction of the trainee using the health care services can be remarkably increased.

Returning back to FIG. 1 and continuing the description according to the present invention, after the step S10 is performed, an evaluation result collection step S20 of providing a search result of the training center to the trainer account, and collecting an evaluation result for training details performed by a trainee from a terminal of the training center determined as generating record of a visit record of the trainee account, may be performed.

In other words, the above-described step S20 may be understood that an on-premise type of service structure may be provided to perform the process of collecting data on training details of the trainee and evaluation results on the training details from a terminal of each training center, and synchronizing the collected data to the cloud server in real time.

In addition, in the above-described step S20, various embodiments may be provided to determine whether a visit record of the trainee account is generated. As one example, after the search results on training centers are provided, it is determined whether a reservation for use of the trainee terminal is present. When the reservation for use is present, it may detect whether the trainee terminal actually has accessed the training center reserved on the scheduled visit date, and a visit history may be generated when the access is detected.

In addition, as another embodiment according to the present invention, each training center may be provided with a terminal for collecting the visit history of the trainee account, to allow the trainee account to perform QR authentication or the like so that visit records of the trainee account may be managed. More preferably, each training tool used by the trainee at a training center may be subject to QR authentication in advance, so that the collection of the training history of the trainee can be facilitated.

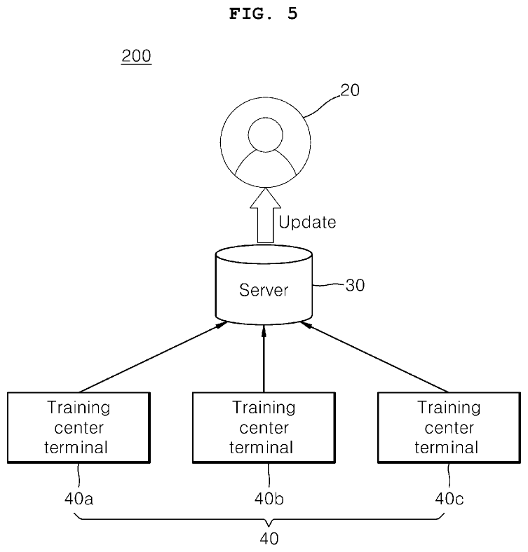

In other words, according to the present invention, due to the above-described determination of whether to generate the visit record for the trainee account, terminals 40a, 40b and 40c of the training centers generating the visit records and a cloud server 30 are connected to each other on a network basis as shown in 200 of FIG. 5, so that training details performed by the trainee and evaluation results on the training details may be collected. Thus, even when the trainee performs the trainings by using multiple training centers, the degree of achievement of the training curriculum and the degree of training performance of the trainee account 20 may be synchronized in real time with the cloud server 30, so as to integrally provide the training status of trainee, so that the convenience of monitoring and managing the training status of trainee can be remarkably increased.

In addition, the terminal of the training center may be preferably understood as equipment equipped with a cognitive ability training program (for example, Tion Plus). In other words, according to the present invention, it may be understood that training data for each trainee may generated by the terminal of the training center.

Returning back to FIG. 1 and continuing the description of step S20 in more detail, it is desirable to pend the collection of evaluation results, when the trainee may be determined as having visited the training center included in the first search result and performed the training, but when a training set of the unit training session scheduled in the training curriculum at the training center has been finished in an incomplete state.

In other words, according to the present invention, the collection of evaluation results may be pended when a situation, such as a trainee's inevitable giving up or stopping of a training at a training center, is detected. In addition, when the trainee requests to search for the next training center, a training center matching the training set of the unit training session finished in an incomplete state may be provided as a second search result, in addition to considering the above-described location information and training performance information, so that evaluation results on the pending training set may be collected and simultaneously search results on training centers capable of performing a training of a next unit training session may be provided.

When the cloud server determines that the above-described trainee has visited a training center included in the second search result, the cloud server may transmit an instruction of performing in advance the training set of the unit training session finished in the incomplete state in the trainer account to a terminal of the training center, to perform collection of the pending evaluation results and then allow the training corresponding to a next training session in the training curriculum set in the trainee account to proceed.

In other words, according to the present invention in the above manner, the trainee is allowed to faithfully conduct the training content for each unit training session in the planned training curriculum, so that the evaluation results on the trainee derived from the evaluation result collection unit may be managed as reliable information.

Next, after performing step S20, an update step S30, of updating the degree of achievement of training curriculum set in the trainee account and the degree of training performance previously stored in the trainee account by using the collected evaluation result, may be performed.

Specifically, the degree of achievement of training curriculum may be understood as a value that is updated in the trainee account by determining whether a training of one unit training session has been completely performed, and the degree of training performance may be a value obtained by quantifying the improvement of the trainee cognitive ability according to the training evaluation result.

In other words, in step S30, it may be understood that the results, on whether the cognitive ability is improved by carrying out the training curriculum set in the trainee account, may be derived from the training details conducted by the trainee in a plurality of training centers and the evaluation results of the training details.

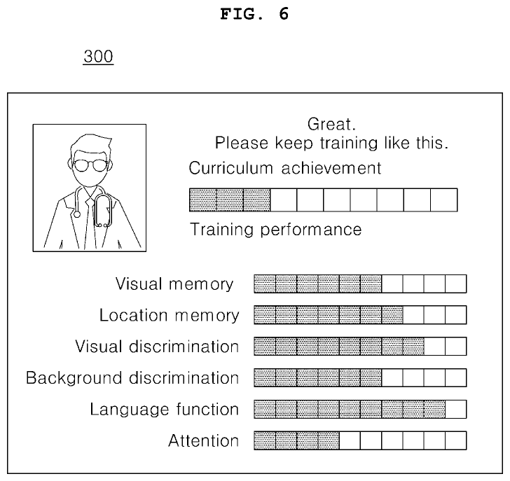

Preferably, the derivation of the training results may be processed and provided as visualized information, and the example is schematically shown in 300 of FIG. 6. The function may be performed, so that trainees can intuitively identify training results on themselves.

Meanwhile, preferably, it may be understood that the training results may be provided to the trainee account as well as viewed and inquired from at least one guardian account registered in the trainee account. However, the present invention is not limited thereto.

Further, it may be understood that at least one data processed in steps S10, S20 and S30 described above in the present invention may be managed through a cloud server, which is a physical or virtual infrastructure linked with the health care application. Accordingly, flexibility and expandability of the service provided in the present invention can be greatly improved.

On the other hand, although not explicitly shown in the accompanying drawings, the health care application mentioned in the present invention may support social network services so as to form a human network between the trainee account and other trainee accounts using the health care application. Further, training curricula of trainees may be compared with each other and trainees with a threshold similarity (for example, 70%) or higher may be grouped as rivals so as to share the evaluation results, so that the training efficiency may be increased. However, the present invention is not limited thereto.

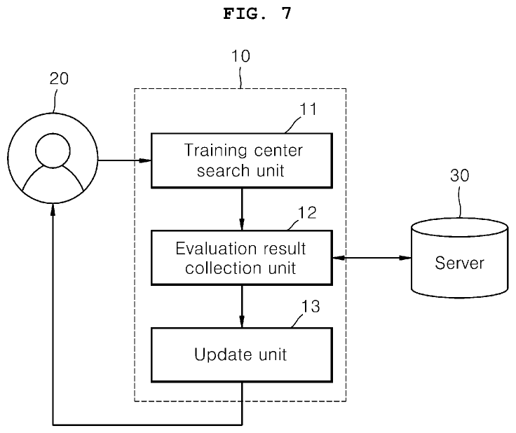

Next, see FIG. 7. FIG. 7 shows an example of a configuration diagram for an apparatus 10 of a senior cloud service for health care according to one embodiment of the present invention. In the following description, unnecessary descriptions for embodiments redundant with those of FIGS. 1 to 6 will be omitted.

As shown in FIG. 7, the apparatus according to the present invention may include a training center search unit 11, an evaluation result collection unit 12 and an update unit 13.

The training center search unit 11, when a health care service request is received from a trainee account through a health care application installed on a trainee terminal 20, functions to search for at least one training center for performing a training scheduled in a training curriculum set in the trainee account 20 based on location information of the trainee terminal.

In other words, the training center search unit 11 may be understood that all functions performed in step S10 of FIG. 1 can be performed. According to the present invention, the function of the training center search unit 11 is performed, so that a training center capable of performing a cognitive ability improvement training by using the surrounding infrastructure can be provided when the trainee wants to train at any time without being restricted by time and space.

Meanwhile, the evaluation result collection unit 12 may function to provide the search results of the training center to the trainer account 20, and collect the evaluation results on training details performed by the trainee from the terminal of the training center determined as having generated a visit record of the trainer account 20.

In other words, the evaluation result collection unit 12 may be understood that all functions performed in step S20 of FIG. 1 can be performed. According to the present invention, the function of the evaluation result collection unit 12 is performed, so that the ease of collecting the training details of the trainee can be increased. In addition, according to the present invention, when the performance of the training set included in the unit training session is inevitably stopped, the evaluation result collection unit 12 may derive the evaluation results after the pending training set is performed in advance in the next training session, so that evaluation reliability according to the training set performed by the trainee can be remarkably increased.

Meanwhile, the update unit 13 may serve to update the degree of achievement of training curriculum set in the trainee account 20 and the degree of training performance previously stored in the trainee account 20 by using the collected evaluation result.

In other words, the update unit 13 may be understood that all functions performed in step S30 of FIG. 1 can be performed. According to the present invention, the function of the update unit 13 is performed, so that the results, on whether the cognitive ability is improved by carrying out the training curriculum set in the trainee account 20, can be derived from the training details conducted by the trainee in a plurality of training centers and the evaluation results of the training details.

Likewise, it may be preferable to understand that processing data in the training center search unit 11, the evaluation result collection unit 12 and the update unit 13 may be managed in the aforementioned cloud server. Accordingly, flexibility and expandability of the service implemented in the present invention can be remarkably improved.

Comprehensively, according to one embodiment of the present invention, the health care application installed on the trainee terminal is used, so that the health care services utilizing on-site infrastructure near the trainee terminal can be provided.

In addition, according to the present invention, the details and evaluation results of the trainee are collected from the terminals of the training center and synchronized with the cloud server in real time, so that the cloud-based health care services, such as unifying and providing multiple databases at a quick response speed, can be provided.

In addition, according to one embodiment of the present invention, places for using health care services may be guided while considering a trainee's preset training curriculum to increase accessibility to the health care services, so that the efficiency of improving the cognitive ability of the trainee can be increased, and the convenience for the trainee using the training center can be remarkably increased.

Although the above embodiments have been described with reference to the limited embodiments and drawings, however, it will be understood by those skilled in the art that various changes and modifications may be made from the above-mentioned description

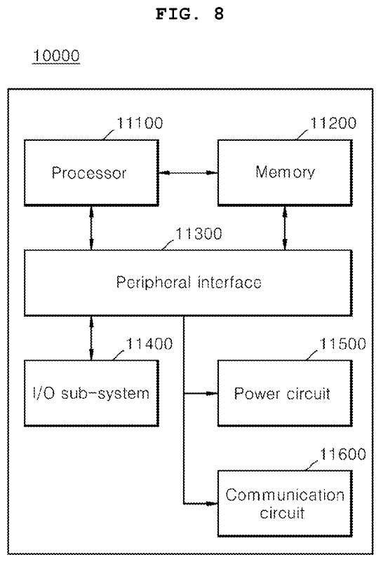

Next, see FIG. 8. FIG. 8 shows one example of an internal configuration of a computing device according to one embodiment of the present invention. In the following description, unnecessary descriptions for embodiments redundant with those of FIGS. 1 to 7 will be omitted.

As shown in FIG. 8, the computing device 10000 may at least include at least one processor 11100, a memory 11200, a peripheral device interface 11300, an input/output subsystem (I/O subsystem) 11400, a power circuit 11500, and a communication circuit 11600. The computing device 10000 may correspond to a user terminal A connected to a tactile interface device or correspond to the above-mentioned computing device B.

The memory 11200 may include, for example, a high-speed random access memory, a magnetic disk, an SRAM, a DRAM, a ROM, a flash memory, or a non-volatile memory. The memory 11200 may include a software module, an instruction set, or other various data necessary for the operation of the computing device 10000.

The access to the memory 11200 from other components of the processor 11100 or the peripheral interface 11300, may be controlled by the processor 11100.

The peripheral interface 11300 may combine an input and/or output peripheral device of the computing device 10000 to the processor 11100 and the memory 11200. The processor 11100 may execute the software module or the instruction set stored in memory 11200, thereby performing various functions for the computing device 10000 and processing data.

The input/output subsystem 11400 may combine various input/output peripheral devices to the peripheral interface 11300. For example, the input/output subsystem 11400 may include a controller for combining the peripheral device such as monitor, keyboard, mouse, printer, or a touch screen or sensor, if needed, to the peripheral interface 11300. According to another aspect, the input/output peripheral devices may be combined to the peripheral interface 11300 without passing through the I/O subsystem 11400.

The power circuit 11500 may provide power to all or a portion of the components of the terminal. For example, the power circuit 11500 may include a power failure detection circuit, a power converter or inverter, a power status indicator, a power failure detection circuit, a power converter or inverter, a power status indicator, or arbitrary other components for generating, managing, or distributing power.

The communication circuit 11600 may use at least one external port to enable communication with other computing devices.

Alternatively, as described above, the communication circuit 11600 may include an RF circuit, if needed, to transmit and receive an RF signal, also known as an electromagnetic signal, thereby enabling communication with other computing devices.

The above embodiment of FIG. 8 is merely an example of the computing device 10000, and the computing device 11000 may have a configuration or arrangement in which some components shown in FIG. 8 are omitted, additional components not shown in FIG. 8 are further provided, or at least two components are combined. For example, a computing device for a communication terminal in a mobile environment may further include a touch screen, a sensor or the like, in addition to the components shown in FIG. 8. The communication circuit 1160 may include a circuit for RF communication of various communication schemes (such as WiFi, 3G, LTE, Bluetooth, NFC, and Zigbee). The components that may be included in the computing device 10000 may be implemented by hardware, software, or a combination of both hardware and software which include at least one integrated circuit specialized in a signal processing or an application.

The methods according to the embodiments of the present invention may be implemented in the form of program instructions to be executed through various computing devices so as to be recorded in a computer-readable medium. In particular, a program according to the embodiment of the present invention may be configured as a PC-based program or an application dedicated to a mobile terminal. The application to which the present invention is applied may be installed in a user terminal through a file provided by a file distribution system. For example, a file distribution system may include a file transmission unit (not shown) that transmits the file according to the request of the user terminal.

The above-mentioned device may be implemented by hardware components, software components, and/or a combination of hardware components and software components. For example, the devices and components described in the embodiments may be implemented by using at least one general purpose computer or special purpose computer such as a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and at least one software application executed on the operating system.

In addition, the processing device may access, store, manipulate, process, and create data in response to the execution of the software. For the further understanding, in some cases, one processing device may be used, however, those skilled in the art will be appreciated that the processing device may include a plurality of processing elements and/or a plurality of types of processing elements. For example, the processing device may include a plurality of processors or one processor and one controller. In addition, other processing configurations, such as a parallel processor, are also possible.

The software may include a computer program, a code, an instruction, or a combination of at least one thereof, may configure the processing device to operate as desired, or may instruct the processing device independently or collectively. In order to be interpreted by the processor or to provide instructions or data to the processor, the software and/or data may be permanently or temporarily embodied in any type of machine, component, physical device, virtual equipment, and computer storage medium or device. The software may be distributed over computing devices connected to networks, so as to be stored or executed in a distributed manner. The software and data may be stored in at least one computer-readable recording medium.

The method according to the embodiment may be implemented in the form of program instructions to be executed through various computing mechanisms so as to be recorded in a computer-readable medium. The computer-readable medium may include program instructions, data files, data structures, and the like, independently or in combination thereof. The program instructions recorded on the medium may be specially designed and configured for the embodiment, or may be known to those skilled in the art of computer software so as to be used. An example of the computer-readable medium includes a magnetic medium such as a hard disk, a floppy disk and a magnetic tape, an optical medium such as a CD-ROM and a DVD, a magneto-optical medium such as a floptical disk, and a hardware device specially configured to store and execute a program instruction such as ROM, RAM, and flash memory.

An example of the program instruction includes a high-level language code to be executed by a computer using an interpreter or the like, as well as a machine code created by a compiler. The above hardware device may be configured to operate as at least one software module to perform the operations of the embodiments, and vise versa.

Although the above embodiments have been described with reference to the limited embodiments and drawings, however, it will be understood by those skilled in the art that various changes and modifications may be made from the above-mentioned description appropriate results may be achieved even though, for example, the described descriptions may be performed in an order different from the described manner, and/or the described components such as system, structure, device, and circuit may be coupled or combined in a form different from the described manner, or replaced or substituted by other components or equivalents. Therefore, other implementations, other embodiments, and equivalents to the claims are also within the scope of the following claims.

What is claimed is:

1. A method of a senior cloud service for health care implemented by a computing device including at least one processor and at least one memory for storing instructions executable in the processor, the method comprising:
a training center search step of searching for at least one training center for performing a training scheduled in a curriculum set in a trainee account based on location information of a trainee terminal, when a health care service request is received from the trainee account through a health care application installed on the trainee terminal;
an evaluation result collection step of providing a search result on the training center to the trainer account, and collecting an evaluation result for training details performed by a trainee from a terminal of the training center determined as creating record of a visit record of the trainee account; and
an update step of updating a degree of achievement of training curriculum preset in the trainee account and a degree of training performance previously stored in the trainee account by using the collected evaluation result, wherein
at least one data processed in the training center search step, the evaluation result collection step, and the update step is managed through a cloud server linked to the health care application.

2. The method of claim 1, wherein the training center search step includes:
giving a weight to location information including at least one of a current location of a terminal accessing the trainee account and a location selected in the trainee account, and training performance information to be obtained from the trainee account to provide a matching training center as a first search result, in which the training performance information is an input value for a training set of a unit training session scheduled in the training curriculum.

3. The method of claim 2, wherein the evaluation result collection step includes:
pending collection of the evaluation result when the trainee is determined as having visited a training center included in the first search result and performed a training, but a training set of the unit training session scheduled in the training curriculum at the training center is determined as having been finished in an incomplete state; and
providing the training center, which match the location information, the training performance information and the training set of the unit training session finished in the incomplete state, as a second search result, upon next training center search step.

4. The method of claim 3, wherein, when the trainee is determined as having visited a training center included in the second search result, the cloud server transmits an instruction of performing in advance the training set of the unit training session finished in the incomplete state in the trainer account to a terminal of the training center to perform collection of the pending evaluation results and then allow training corresponding to a next training session in the training curriculum set in the trainee account to proceed.

5. The method of claim 1, wherein the training center search step, when a plurality of trainings are scheduled in the training curriculum set in the trainee account, and a search result for a single training center capable of performing the training set is not found, includes:
providing search results grouped by two or more training centers by searching for two or more training centers capable of performing an individual training included in the training sets.

6. The method of claim 5, wherein an accessibility index from the trainer terminal is indicated in the search results grouped into the two or more training centers, in which the accessibility index is indicated preferentially in the trainer terminal by applying a higher weight when a number of training centers required to be used by the trainee is smaller and when a moving distance between the training centers is shorter.

7. An apparatus of a senior cloud service for health care implemented by a computing device including at least one processor and at least one memory for storing instructions executable in the processor, the apparatus comprising:
a training center search unit for searching for at least one training center for performing a training scheduled in a curriculum set in a trainee account based on location information of a trainee terminal, when a health care service request is received from the trainee account through a health care application installed on the trainee terminal;
an evaluation result collection unit for providing a search result on the training center to the trainer account, and collecting an evaluation result for training details performed by a trainee from a terminal of the training center determined as creating record of a visit record of the trainee account; and
an update unit for updating a degree of achievement of training curriculum preset in the trainee account and a degree of training performance previously stored in the trainee account by using the collected evaluation result, wherein
at least one data processed in the training center search unit, the evaluation result collection unit, and the update unit is managed through a cloud server linked to the health care application.

8. A computer-readable recording medium that stores instructions for allowing a computing device to perform steps comprising:
a training center search step of searching for at least one training center for performing a training scheduled in a curriculum set in a trainee account based on location information of a trainee terminal, when a health care service request is received from the trainee account through a health care application installed on the trainee terminal;
an evaluation result collection step of providing a search result on the training center to the trainer account, and collecting an evaluation result for training details performed by a trainee from a terminal of the training center determined as creating record of a visit record of the trainee account; and
an update step of updating a degree of achievement of training curriculum preset in the trainee account and a degree of training performance previously stored in the trainee account by using the collected evaluation result, wherein
at least one data processed in the training center search step, the evaluation result collection step, and the update step is managed through a cloud server linked to the health care application.
